(19) United States
(12) Patent Application Publication
(10) Pub. No.: US 2025/0382307 A1
(43) Pub. Date: Dec. 18, 2025

(54) IMPROVED METHODS FOR PREPARING N-METHYL ALANINE ESTERS OF MAYTANSINOL

(71) Applicant: IMMUNOGEN, INC., Waltham, MA (US)

(72) Inventors: Feng Liang, South Plainfield, NJ (US); Lynette Oh, Riehen (CH)

(21) Appl. No.: 19/112,080

(22) PCT Filed: Sep. 15, 2023
(86) PCT No.: PCT/US2023/032874

§ 371 Date: Mar. 14, 2025

Related U.S. Application Data
(60) Provisional application No. 63/407,380, filed on Sep. 16, 2022.

(51) Int. Cl. C07D 498/18

(52) U.S. Cl. CPC C07D 498/18

(57) ABSTRACT

Disclosed is a method of preparing an amino acid ester of maytansinol by reacting maytansinol with an N-carboxyanhydride of an amino acid (NCA) in the presence of a proton sponge and a Lewis acid.

### RELATED APPLICATION

This application claims priority to U.S. Provisional Application No. 63/407,380, filed on Sep. 16, 2022, the entire content of which is incorporated herein by reference.

### FIELD OF THE INVENTION

The present invention relates to improved methods for acylating maytansinol to prepare N-methyl alanine esters of maytansinol (MayNMA).

### BACKGROUND OF THE INVENTION

Maytansinoids are highly cytotoxic compounds, including maytansinol and C-3 esters of maytansinol (U.S. Pat. No. 4,151,042). Amino acid esters of maytansinol are valuable intermediates that can be coupled to carboxylic acids to provide maytansinoids. Among the C-3 amino acid esters of maytansinol, N-methyl-alanine esters of maytansinol (MayNMA) has been shown as very useful precursors for a variety of maytansinoid derivatives that can be used for preparing anti-cancer drugs.

MayNMA is usually prepared via acylation reaction of maytansinol with an N-carboxyanhydride (NCA). A significant disadvantage of the acylation reaction is that it also forms a by-product comprising an extra N-methyl-alanyl moiety in the C3 side chain, referred to as “extra-NMA” or “May(NMA)₂”. May(NMA)₂ is difficult to separate from MayNMA and can cause formation of other impurities if not completely removed.

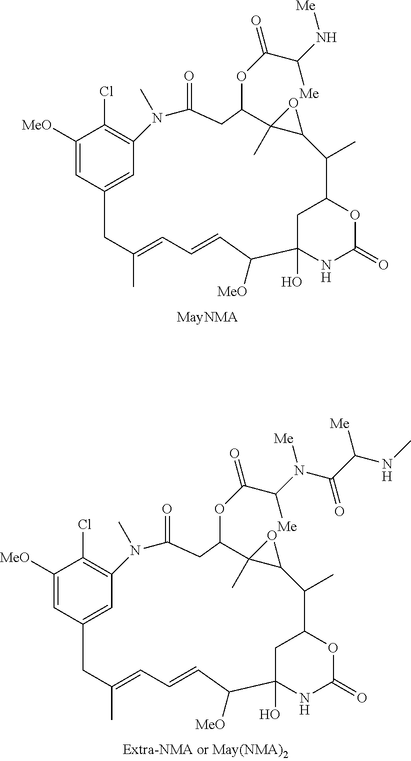 [SMILES: CNC(C)C(=O)OC1CC(=O)N(C)c2cc(cc(OC)c2Cl)C/C(C)=C/C=C/C(OC)C2(O)CC(OC(=O)N2)C(C)C2OC12C ; CNC(C)C(=O)N(C)C(C)C(=O)OC1CC(=O)N(C)c2cc(cc(OC)c2Cl)C/C(C)=C/C=C/C(OC)C2(O)CC(OC(=O)N2)C(C)C2OC12C]

There is a need to improve the yield and robustness of the processes to prepare MayNMA and to minimize by-products formed, especially those that are difficult to remove.

### SUMMARY OF THE INVENTION

The present invention provides new reaction conditions for acylating maytansinol with better control of impurities and purification procedures, which are more efficient and suitable for large scale manufacturing. The methods disclosed herein significantly reduce the equivalents of Lewis acid required for the acylation reaction and improve stereoisomer selectivity by using proton sponge as a base. New work up conditions are also provided herein, which can quench unreacted N-carboxyanhydride (NCA) more effectively and reduce the formation of May(NMA)₂.

In one aspect, the present invention relates to a method of preparing a compound represented by Formula (I):

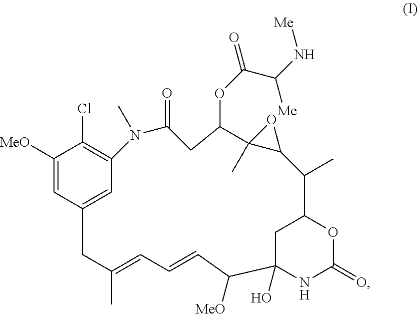 [SMILES: CNC(C)C(=O)OC1CC(=O)N(C)c2cc(cc(OC)c2Cl)C/C(C)=C/C=C/C(OC)C2(O)CC(OC(=O)N2)C(C)C2OC12C]

or a salt thereof, comprising reacting maytansinol with an N-carboxyanhydride in a reaction mixture comprising a base and a Lewis acid to form the compound of Formula (I) or a salt thereof, wherein the N-carboxyanhydride is represented by Formula (II):

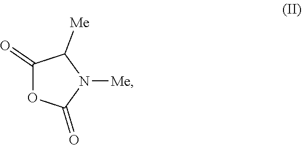

and the base is proton sponge.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents which may be included within the scope of the present invention as defined by the claims. One skilled in the art will recognize many methods and materials similar or equivalent to those described herein, which could be used in the practice of the present invention.

It should be understood that any of the embodiments described herein can be combined with one or more other embodiments of the invention, unless explicitly disclaimed or improper. Combination of embodiments are not limited to those specific combinations claimed via the multiple dependent claims.

### Definitions

Unless otherwise defined herein, scientific and technical terms used in this application shall have the meanings that are commonly understood by those of ordinary skill in the art to which this invention belongs. Generally, nomenclature used in connection with the compounds, composition and methods described herein, are those well-known and commonly used in the art.

Chemistry terms used herein are used according to conventional usage in the art, as exemplified by “The McGraw-Hill Dictionary of Chemical Terms”, Parker S., Ed., McGraw-Hill, San Francisco, C.A. (1985).

The term “herein” means the entire application.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “including” is used to mean “including but not limited to.” “Including” and “including but not limited to” are used interchangeably.

As used herein, “about” or “approximately” means within an acceptable error range for the particular value as determined by one of ordinary skill in the art, which will depend in part on how the value is measured or determined, i.e., the limitations of the measurement system.

The use of the terms “a” and “an” and “the” and similar referents in the context of describing the elements (especially in the context of the following claims) are to be construed to cover both the singular and the plural, unless otherwise indicated herein or clearly contradicted by context.

The term “or” as used herein should be understood to mean “and/or,” unless the context clearly indicates otherwise.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The term “compound” is intended to include compounds for which a structure or formula or any derivative thereof has been disclosed in the present invention or a structure or formula or any derivative thereof that has been incorporated by reference. The term also includes, stereoisomers, geometric isomers, tautomers, solvates, and salts (e.g., pharmaceutically acceptable salts) of a compound of all the formulae disclosed in the present invention. The term also includes any solvates, hydrates, and polymorphs of any of the foregoing. The specific recitation of “stereoisomers,” “geometric isomers,” “tautomers,” “solvates,” “salt” “hydrate,” or “polymorph” in certain aspects of the invention described in this application shall not be interpreted as an intended omission of these forms in other aspects of the invention where the term “compound” is used without recitation of these other forms.

The term “chiral” refers to molecules that have the property of non-superimposability of the mirror image partner, while the term “achiral” refers to molecules that are superimposable on their mirror image partner.

The term “stereoisomer” refers to compounds that have identical chemical constitution and connectivity, but different orientations of their atoms in space that cannot be interconverted by rotation about single bonds.

The term “diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers can separate under high resolution analytical procedures such as crystallization, electrophoresis and chromatography.

The term “enantiomers” refer to two stereoisomers of a compound that are non-superimposable mirror images of one another. Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds,” John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

The term “tautomer” or “tautomeric form” refers to structural isomers of different energies that are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

The term “salt” as used herein, refers to an organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A salt can involve the inclusion of another molecule such as an acetate ion, a succinate ion or other counter ion. The counter ion can be any organic or inorganic moiety that stabilizes the charge on the parent compound. Furthermore, a salt can have more than one charged atom in its structure. Instances where multiple charged atoms are part of the salt can have multiple counter ions. Hence, a salt can have one or more charged atoms and/or one or more counter ion.

If the compound of the invention is a base, the desired salt can be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of the invention is an acid, the desired salt can be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as triethylamine, piperidine, morpholine and piperazine, and inorganic salts derived from sodium, calcium, potassium, magnesium, manganese, iron, copper, zinc, aluminum and lithium.

In certain embodiments, the salt is a pharmaceutically acceptable salt. The phrase “pharmaceutically acceptable” indicates that the substance or composition must be compatible chemically and/or toxicologically, with the other ingredients comprising a formulation, and/or the mammal being treated therewith.

The term “solvate” means a compound that further includes a stoichiometric or non-stoichiometric amount of solvent such as water, isopropanol, acetone, ethanol, methanol, DMSO, ethyl acetate, acetic acid, and ethanolamine dichloromethane, 2-propanol, or the like, bound by non-covalent intermolecular forces. Solvates or hydrates of the compounds are readily prepared by addition of at least one molar equivalent of a hydroxylic solvent such as methanol, ethanol, 1-propanol, 2-propanol or water to the compound to result in solvation or hydration of the imine moiety.

The term “precursor” of a given group refers to any group which may lead to that group by any deprotection, a chemical modification, or a coupling reaction.

The term “amino acid” refers to naturally occurring amino acids or non-naturally occurring amino acid. In some embodiments, the amino acid is represented by NH₂—C(R^{aa′}R^{aa})—C(═O)OH, wherein R^{aa} and R^{aa′} are each independently H, an optionally substituted linear, branched or cyclic alkyl, alkenyl or alkynyl having 1 to 10 carbon atoms, aryl, heteroaryl or heterocyclyl or R^{aa} and the N-terminal nitrogen atom can together form a heterocyclic ring (e.g., as in proline). The term “amino acid residue” refers to the corresponding residue when one hydrogen atom is removed from the amine and/or carboxy end of the amino acid, such as —NH—C(R^{aa′}R^{aa})—C(═O)—.

The term “cation” refers to an ion with positive charge. The cation can be monovalent (e.g., Na⁺, K⁺, etc.), bi-valent (e.g., Ca²⁺, Mg²⁺, etc.) or multi-valent (e.g., Al³⁺ etc.). Preferably, the cation is monovalent.

The term “base” refers to a substance that can accept a hydrogen ion (proton) or donate a pair of valence electrons. Examples of the suitable bases include imidazole, piperidine, 4-methylpiperidine, tetramethylpiperidine, morpholine, N-methylmorpholine, pyridine, 2,6-lutidine, dimethylformamide, piperazine, pyrrolidine, 1-methylpyrrolidine, 1,8-diazabicyclo[5.4.0]undec-7-ene (DBU), diethylamine (DEA), a trialkylamine (e.g., N,N-diisopropylethylamine (DIPEA), triethylamine (TEA), and 1,8-Diazabicycloundec-7-ene), a metal alkoxide (e.g., sodium tert-butoxide and potassium tert-butoxide), an alkyl metal (e.g., tert-butyllithium, methyl lithium, n-butyl lithium, tert-butyl lithium, lithium di-isopropylamide, pentyl sodium, and 2-phenyl isopropyl-potassium), an aryl metal (e.g., phenyl lithium), a metal hydride (e.g., sodium hydride), a metal amide (e.g., sodium amide, potassium amide, lithium diisopropylamide and lithium tetramethylpiperidide), and a silicon-based amide (e.g., sodium bis(trimethylsilyl)amide and potassium bis(trimethylsilyl)amide).

The term “proton sponge” refers to a base with very high basicity and contains two amino groups or derivatives thereof, in which the two amino groups or derivatives thereof are in sterically strained position. In some embodiments, the proton sponge is 1,8-bis(dimethylamino)naphthalene or a derivative thereof.

The term “acid” refers to a substance that can donate a hydrogen ion (proton) or form a covalent bond with an electron pair. Examples of acids include the inorganic substances known as the mineral acids—sulfuric, nitric, hydrochloric, and phosphoric acids—and the organic compounds belonging to the carboxylic acid, sulfonic acid, and phenol groups. Such substances contain one or more hydrogen atoms that, in solution, are released as positively charged hydrogen ions. Examples of acid includes formic acid, acetic acid, trifluoroacetic acid (TFA), pyridinium p-toluenesulfonate (PPTS), p-toluenesulfonic acid, methanesulfonic acid, camphorsulfonic acid, phosphoric acid, sulfuric acid, hydrochloric acid (HCl), and trichloroacetic acid.

The term “Lewis acid” refers to an acid substance which can employ an electron lone pair from another molecule in completing the stable group of one of its own atoms. Exemplary Lewis acids for use in the disclosed methods include boron trifluoride etherate (BF₃·OEt₂), zinc triflate, zinc chloride, magnesium bromide, magnesium triflate, copper triflate, copper (II) bromide, copper (II) chloride, magnesium chloride, and aluminum chloride (AlCl₃).

The term “drying agent” refers to an agent that can remove water from a solution. Examples of a suitable drying agent include, but are not limited to, molecular sieves, sodium sulfate, calcium sulfate, calcium chloride, and magnesium sulfate. The physical forms of the drying agents include, but are not limited to, granular beads or powders. Preferably, the drying agent is molecular sieve. Alternatively, the drying agent is sodium sulfate.

The term “nucleophilic reagent” refers to a reactant that reacts with electropositive centers in the N-carboxyanhydride represented by Formula (II) to decompose the N-carboxyanhydride. Examples of suitable nucleophilic reagent include water, an alcohol (methanol, ethanol, n-propanol, isopropanol, or tert-butanol) and a primary or secondary amine (e.g., methylamine, ethylamine, dimethylamine, diethylamine, etc.). Preferably, the nucleophilic reagent is an alcohol. Alternatively, the nucleophilic reagent is water.

The term “precipitation” refers to the process of transforming a dissolved substance into an insoluble solid from a solution comprising the substance (e.g., saturated solution of the substance). The solid formed is called the precipitate. The clear liquid remaining above the precipitated or the centrifuged solid phase is also called the ‘supernate’ or ‘supernatant’. In some embodiments, precipitation can occur by adding a co-solvent, in which the substance has low or no solubility, to a solution of the substance. In some embodiments, cooling a solution comprising the substance can result in precipitation.

The term “organic solvent” refers to carbon-based substances capable of dissolving or dispersing one or more other substances. Many classes of chemicals are used as organic solvents, including aliphatic hydrocarbons, aromatic hydrocarbons, amines, esters, ethers, ketones, and nitrated or chlorinated hydrocarbons. Exemplary organic solvents include dichloromethane (CH₂Cl₂ or DCM), dichloroethane (DCE), acetonitrile (ACN or MeCN), ethyl acetate, methanol (MeOH), ethanol, tetrahydrofuran (THF), toluene, N-methylmorpholine (NMM), dimethylformamide (DMF), dimethyl sulfoxide (DMSO), dimethylacetamide (DMA or DMAc), or any combination thereof.

### Methods of the Present Invention

The methods disclosed herein replace N,N-diisopropylethyl amine (DIPEA) used in the previously-disclosed procedures with proton sponge, which has better stereoisomer selectivity and significantly reduces epimerization during the acylation reaction. The equivalent of Lewis acid (e.g., zinc triflate) based on maytansinol is significantly reduced (e.g., reduced from 5.6 equivalent to 1.5 equivalent). New quench conditions (e.g., quench with a solution of ammonia in THF) are used to react with excess N-carboxyanhydride (NCA). NCA can lead to the formation of significant amounts of May(NMA)₂ impurity when unquenched. The new quench conditions do not degrade the product MayNMA and therefore, immediate work-up after the quenching reaction is not absolutely required. In contrast, previous quench conditions may negatively impact the quality of final product MayNMA if the quenching reaction is not immediately followed with work-up procedure to isolate the MayNMA product from the reaction mixture.

In a first embodiment, the present invention provides a method of preparing a compound represented by Formula (I):

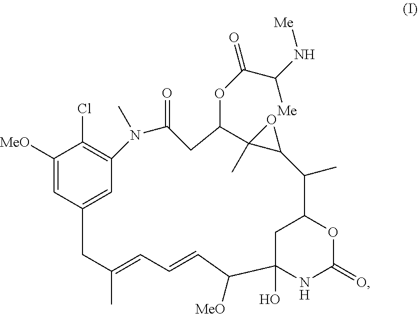 [SMILES: CNC(C)C(=O)OC1CC(=O)N(C)c2cc(cc(OC)c2Cl)C/C(C)=C/C=C/C(OC)C2(O)CC(OC(=O)N2)C(C)C2OC12C]

or a salt thereof, comprising reacting maytansinol with an N-carboxyanhydride in a reaction mixture comprising a base and a Lewis acid to form the compound of Formula (I) or a salt thereof, wherein the N-carboxyanhydride is represented by Formula (II):

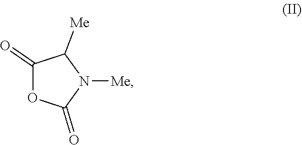

and the base is proton sponge.

In a 1^{st} specific embodiment, the compound of Formula (I) is represented by Formula (Ia):

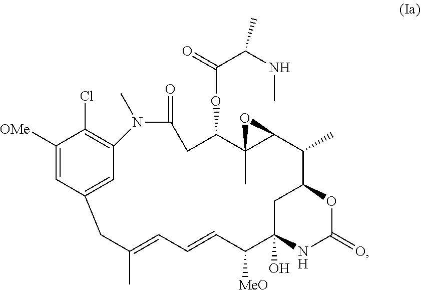 [SMILES: CN[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

and
the N-carboxyanhydride of Formula (I) is represented by Formula (IIa):

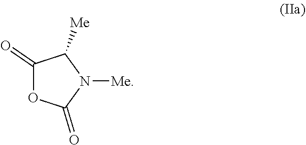

In some embodiments, the proton sponge is N,N,N′N′-tetramethyl-1,8-naphthalenediamine, 2,7-dibromo-1,8-bis(dimethylamino)naphthalene (Br₂DMAN), 1,8-bis(hexamethyltriaminophosphazenyl)naphthalene (HMPN), 2,7-di(4-tolylethynyl)-1,8-bis(dimethylamino)naphthalene or its cation, 2,4-bis(trifluoroacetyl)-1,8-bis(dimethylamino)naphthalene, or 2,6-difluoro-1,3,4,5,7,8-hexakis(dimethylamino)naphthalene. In a specific embodiment, the proton sponge is N,N,N′,N′-tetramethyl-1,8-naphthalenediamine.

Any suitable amount of proton sponge (e.g., N,N,N′,N′-tetramethyl-1,8-naphthalenediamine) can be used. In some embodiments, the molar ratio of the proton sponge (e.g., N,N,N′,N′-tetramethyl-1,8-naphthalenediamine) to maytansinol is in the range of 0.1:1 to 10:1, 1:1 to 5:1, 2:1 to 4:1, or 2.5:1 to 3:1. In a specific embodiment, the molar ratio of the proton sponge (e.g., N,N,N′,N′-tetramethyl-1,8-naphthalenediamine) to maytansinol is 2.75:1.

In some embodiments, the Lewis acid is zinc triflate, zinc chloride, magnesium bromide, magnesium triflate, copper triflate, copper (II) bromide copper (II) chloride, or magnesium chloride. In a specific embodiment, the Lewis acid is zinc triflate.

Any suitable amount of Lewis acid (e.g., zinc triflate) can be used. In some embodiments, the molar ratio of the Lewis acid (e.g., zinc triflate) to maytansinol is in the range of 0.1:1 to 10:1, 0.1:1 to 6:1, 1:1 to 5:1, 1.5:1 to 3:1, or 2:1 to 2.5:1. In a specific embodiment, the molar ratio of the Lewis acid (e.g., zinc triflate) to maytansinol is 1.5:1.

In some embodiments, the reaction mixture further comprises a drying agent. The drying agent is a molecular sieve, sodium sulfate, calcium sulfate, calcium chloride, or magnesium sulfate. In a specific embodiment, the drying agent is a molecular sieve. In a more specific embodiment, the molecular sieve is in the form of granular beads or powders.

Sufficient amounts of drying agents are used to remove dissolved water from the reaction solvent. The quantity of drying agent is not critical, provided that the reaction solution is rendered substantially anhydrous. The drying agent can be used directly in the reaction vessel or by being contained in the vessel by a semi permeable barrier, such as a sintered glass container.

The time required for the reaction can be easily monitored by one skilled in the art using techniques including, but not limited to, high pressure liquid chromatography and thin layer chromatography. A typical reaction is completed after stirring for 24 hours but may be performed at a slower or a faster rate depending on various factors, such as reaction temperature and concentrations of the reactants.

The reaction between maytansinol and the compound of Formula (II) or (IIa) can be carried out in any suitable organic solvent(s). Suitable solvents are readily determined by one of ordinary skill in the art, and include, but are not limited to, DMF, DMSO, THF, CH₂Cl₂, acetonitrile, dichloroethane, dimethylacetamide, methanol, ethanol, and toluene. In a specific embodiment, the solvent is a mixture of DMF and THF.

A volume ratio between 1:20 and 20:1, between 1:10 and 10:1, between 1:3 and 3:1, or between 1:2 and 2:1 of DMF to THF can be used as solvents for the reaction. In a specific embodiment, the volume ratio of DMF to THF is 9:1.

The reaction between maytansinol and the compound of Formula (II) or (IIa) can be carried out at a suitable temperature. In some embodiments, the reaction is carried out at a temperature between −50° C. and 50° C., between −30° C. and 30° C., between −25° C. and 25° C., or between −15° C. and 25° C.

Although equal molar amounts of maytansinol to an N-carboxyanhydride can be used, more commonly N-carboxyanhydride is used in excess. Exemplary molar ratios of maytansinol to N-carboxyanhydride range from 1:1 to 1:10, more commonly 1:2 to 1:7,1:1 to 1:4 or 1:3.5 to 1:4.5. In a specific embodiment, the molar ratio of maytansinol to N-carboxyanhydride is 1:4.

In some embodiments, the methods described herein further comprise quenching unreacted N-carboxyanhydride by contacting the reaction mixture after the reaction of maytansinol and the N-carboxyanhydride with a solution of ammonia in an organic solvent.

The organic solvent can be DMF, DMSO, THF, CH₂Cl₂, acetonitrile, dichloroethane, dimethylacetamide, methanol, ethanol, or toluene. In a specific embodiment, the reaction mixture after the reaction of maytansinol and the N-carboxyanhydride is contacted with a solution of ammonia in THF to quench unreacted N-carboxyanhydride.

The concentration of ammonia in THF is 0.1 M-10 M, 0.1 M-5 M, 0.2 M-1 M, 0.2 M-0.6 M, or 0.3 M-0.5 M. In a specific embodiment, the concentration of ammonia in THF is 0.4 M.

The quench can be carried out at a suitable temperature. In some embodiments, the quench is carried out at a temperature between −100° C. and 100° C., between −50° C. and 80° C., between −40° C. and 60° C., between −30° C. and 40° C., between −30° C. to 25° C., or between −20° C. to −16° C. In a specific embodiment, the quench is carried out at a temperature of −15° C.

In some embodiments, the methods described herein further comprises reacting unreacted N-carboxyanhydride with a nucleophilic reagent. In some embodiments, the nucleophilic reagent is water or an alcohol. Suitable alcohols include, but are not limited to methanol, ethanol, n-propanol, isopropanol, and tert-butanol.

The amount of a nucleophilic reagent can be readily determined by a skilled person in the art. Preferably, a sufficient quantity of nuclophilic reagent is used to quench the unreacted N-carboxyanhydride. In some embodiments, excess quantities of nucleophilic reagent can also be used. A typical reaction is completed after stirring 1 hour but may be performed at a slower or a faster rate depending on various factors, such as temperature.

In some embodiments, the reaction mixture after the reaction of maytansinol and the N-carboxyanhydride is contacted with an aqueous solution containing bicarbonate or carbonate or with a metal scavenger. Preferably, the reaction mixture is reacted with the nucleophilic reagent to quench excess N-carboxyanhydride prior to the reaction mixture being contacted with an aqueous solution containing bicarbonate or carbonate or with a metal scavenger.

Metal scavengers known in the art can be used (see, for example, chapter 9 in “The Power of Functional Resin in Organic Synthesis” by Aubrey Mendoca, Wiley-VCH Verlag GmbH & Co. KGaA, 2008). Examples of metal scavengers include, but are not limited to, polymer and silica-based metal scavenger (e.g., QuadraPure™ and QuadraSil™ by Sigma-Aldrich, SiliaMetS® by SiliCycle, Smopex® by Johnson Matthey and Biotage metal scavengers), carbon-based scavengers (e.g., QuadraPure™ C by Sigma-Aldrich).

All references cited herein and in the examples that follow are expressly incorporated by reference in their entireties.

### EXAMPLES

The following solvents, reagents, protecting groups, moieties and other designations may be referred to by their abbreviations in parenthesis:
DCM dichloromethane
DIPEA N,N-diisopropylethylamine
DMF N,N-dimethylformamide
D-NCA (R)-3,4-dimethyloxazolidine-2,5-dione
EtOAc ethyl acetate
eq molar equivalents
HPLC high performance liquid chromatography
IPAc isopropyl acetate
IPC in-process control
hr(s) Hour(s)
L-MayNMA (1⁴S,1⁶S,3²S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl methyl-L-alaninate
MayOH Maytansinol-(1⁴S,1⁶S,3²S,3³S,2R,4S,10E,12E,14R)-8⁶-chloro-1⁴,4-dihydroxy-8⁵,14-dimethoxy-3³,2,7,10-tetramethyl-7-aza-1(6,4)-oxazinana-3(2,3)-oxirana-8(1,3)-benzenacyclotetradecaphane-10,12-diene-1²,6-dione
MeCN acetonitrile
min(s) minute(s)
MP mobile phase
MTBE tert-butyl methyl ether
NMT not more than
LDPE low density polyethylene
L-NCA (S)-3,4-dimethyloxazolidine-2,5-dione
NaHCO₃sodium bicarbonate
Na₂SO₄ sodium sulfate
T3P Propanephosphonic acid anhydride
r.t. Room temperature
TFA trifluoroacetic acid
THF tetrahydrofuran
UPLC ultra performance liquid chromatography
vol volumes
w.r.t. with respect to
wt weight
° C. degree Celsius

All reactions were performed under nitrogen atmosphere with magnetic stirring. All solvents were purchased as anhydrous solvents from Aldrich. Maytansinol was produced as described (Widdison et al., J. Med. Chem., 49:4392-4408 (2006)). (S)-3,4-Dimethyloxazolidine-2,5-dione was prepared by the method reported in the literature: Guillaume Laconde, Muriel Amblard, and Jean Martinez, Org. Lett., 2021, 23, 6412-6416. Nuclear magnetic resonance (NMR) spectra (¹H 400 MHz, ¹³C 100 MHz) were obtained on a Bruker ADVANCE™ series NMR. HPLC/MS data was obtained using a Bruker ESQUIRE™ 3000 ion trap mass spectrometer in line with an Agilent 1100 series HPLC.

### HPLC Method:

Column: 150×4.6 mm C8, particle size 5 micron, Zorbax P/N 993967-906
Solvents: A deionized water+0.1% TFA
Solvent B: Acetonitrile
Flow rate 1.0 mL/min Temperature: Ambient
Injection volume: 15 μL

Gradient

| Time | % B |
|---|---|
| 0 | 25 |
| 25 | 50 |
| 26 | 95 |
| 30 | 95 |
| 31 | 25 |
| 37 | 25 |

Data was displayed from 0-25 min in HPLC traces.

### Sample Preparation for Analytical HPLC Method:

Aliquots (20 μL) of a given mixture were added to acetonitrile (1.5 mL) in an autosampler vial. The vial was capped and shaken then placed in a 15° C. autosampler. An injection volume (15 μL) was analyzed for each HPLC run.

### Example 1. Synthesis of L-NCA (Compound IIa)

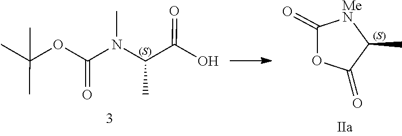

A solution of Boc-N-methyl-L-alanine (Compound 3, 1.0 eq, 1 wt) in ethyl acetate (10 vol) was cooled to 0±5° C. under nitrogen. T3P (50% in EtOAc, 1.13 eq) was slowly added via an addition funnel at such a rate to maintain an internal temperature below 2 t 5° C. The mixture stirred for 5-15 mins at this temperature and was added pyridine (1.0 eq) at 0±5° C. The internal temperature was maintained below 2±5° C. during the addition. The reaction continued to stir for 22-26 hrs at 0±5° C. and was quenched with water (5 vol) at 5±5° C. The organic layer was separated, washed with water (5 vol) at 5±5° C. to remove pyridine and T3P by products, and then washed with 25% sodium chloride solution (5 vol) at 5±5° C. All the organic layers were combined and concentrated under reduced pressure. The residue was re-dissolved in EtOAc (5 vol) and stirred at 25±5° C. Heptane (4 vol) was added to the stirred EtOAc solution to precipitate the product. The solid was filtered, washed with heptane (1 vol) and dried in vacuum oven to afford Compound IIa as white granular solid (achiral HPLC purity≥95.0%; chiral purity D-NCA≤0.6%). A run with 900 g Boc-N-methyl-L-alanine afforded Compound IIa (yield, 53%) with purity by NMR=97% and purity by HPLC=96% (spec).

### Example 2. Synthesis of L-MayNMA (Compound Ia) in the Presence of Proton Sponge and Quench with Ammonium Solution

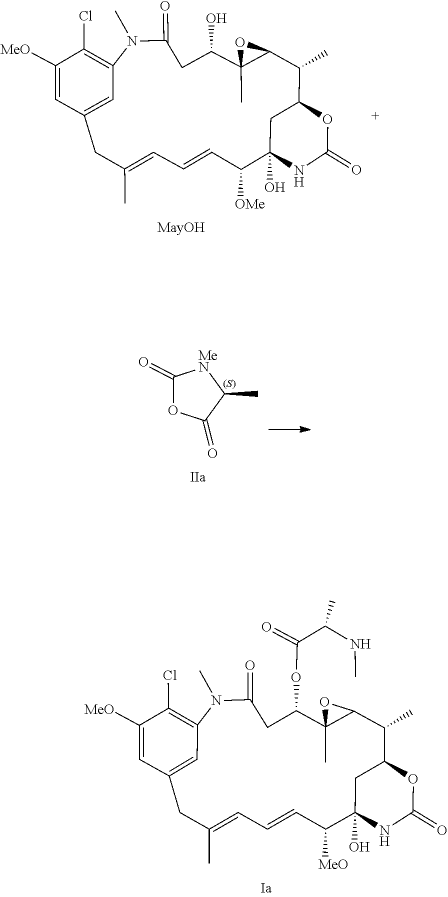

MayOH (1.0 eq, 1 wt), L-NCA (Compound IIa, 4.0 eq), proton sponge (N,N,N′,N′-tetramethyl-1,8-naphthalenediamine, 2.75 eq), and zinc triflate Zn(OTf)₂ (1.5 eq) were dissolved in DMF/THF solution (5.4 vol/0.6 vol) under nitrogen at a temperature<−15° C. and warmed to 10±2.5° C. until all the solids were dissolved. The reaction continued to stir for at least 18 hrs at 22±2° C. and cooled to <−25° C. A saturated solution of ammonia (NH₃) in THF (0.4 M, 22 vol) was added while keeping temperature<−15° C. to quench excess L-NCA. The resultant mixture stirred for 16 hrs at 17±3° C. and was filtered through a pad of celite. The filter cake was washed with MeCN (2×5 vol). The filtrate was concentrated under reduced pressure to an oil while keeping temperature NMT 25° C. The residue was re-dissolved in MeCN (50-100 vol) and added 1:1 acetone/CH₂Cl₂ (5 vol) to precipitate excess proton sponge. The solid was filtered and washed with 1:1 acetone/CH₂Cl₂ (1 vol). The filtrate was transferred to 125 g prepacked silica (on 5 g scale of MayOH) in a column. Elution of the silica column with 1:1 acetone/CH₂Cl₂ to afford L-MayNMA (Compound Ia, yield, 65-75%). The largest scale run using this method is 5 g MayOH. ¹H NMR (400 MHz, d6-DMSO) δ 7.20 (d, J=1.6 Hz, 1H), 7.14 (d, J=1.7 Hz, 1H), 6.88 (s, 1H), 6.57 (dd, J=15.4, 11.1 Hz, 1H), 6.21 (d, J=11.2 Hz, 1H), 5.88 (s, 1H), 5.41 (dd, J=15.4, 9.0 Hz, 1H), 4.64 (dd, J=11.9, 2.8 Hz, 1H), 4.15-4.04 (m, 1H), 3.92 (s, 3H), 3.50 (t, J=10.3 Hz, 2H), 3.39 (p, J=7.3, 6.6 Hz, 1H), 3.33 (s, 3H), 3.26 (d, J=12.7 Hz, 3H), 3.06 (s, 2H), 2.98 (s, 3H), 2.60 (d, J=9.7 Hz, 1H), 2.53-2.42 (m, 2H), 2.17 (s, 3H), 2.04 (dd, J=14.0, 2.9 Hz, 2H), 1.62 (s, 3H), 1.51-1.38 (m, 2H), 1.31 (d, J=12.2 Hz, 1H), 1.20 (d, J=6.7 Hz, 3H), 1.11 (d, J=6.0 Hz, 3H), 1.09 (s, 5H), 0.80 (s, 3H). ¹³C NMR (101 MHz, d6-DMSO) δ 173.60, 168.01, 155.16, 151.18, 141.58, 140.94, 139.08, 132.20, 128.44, 124.58, 122.41, 117.34, 113.86, 88.13, 80.09, 76.08, 73.32, 72.05, 65.36, 60.85, 56.66, 56.55, 56.14, 48.72, 45.73, 38.00, 36.11, 35.11, 32.69, 32.31, 26.82, 16.66, 15.24, 14.55, 11.60. ESI+ (m/z): 650.10.

### Example 3. Comparing Different Quench Conditions

The reaction conditions (Table 1) for preparing L-MayNMA (Compound Ia) were the same as those described in Example 2. At the end of the reaction, quenching with NH₃ in THF was compared against quenching with either sat Na₂CO₃ or water. The IPC results are shown in Table 1 below.

TABLE 1

| | A | B | |
|---|---|---|---|
| IPC before | L-May(NMA)₂: 0.1 | L-May(NMA)₂: 0.1 area % | |
| quench | area % | Impurities: 2.8 area % | |
| | Impurities: 4.0 | MayOH: 1.1 area % | |
| | area % | | |
| | MayOH: 3.0 area % | | |
| Dilution | 10 mL DCM | 30 mL DCM and split in two halves | |
| Dropwise | 0.4M NH₃/THF | Sat. aq. Na₂CO₃ | DI water |
| charged | (22 mL) | (1 mL) | (1 mL) |
| Temperature | Slightly exothermic | Exothermic | |
| Agitated at | L-May(NMA)₂: 0.1 | L-May(NMA)₂: | L-May(NMA)₂: |
| 22° C. | area % | 0.4 area % | 1.8 area % |
| | Impurities: 4.0 | Impurities: 3.1 | Impurities: 3.0 |
| | area % | area % | area % |
| | MayOH: 3.1 | MayOH: 2.3 | MayOH: 3.3 |
| | area % | area % | area % |

Filter, washing cake with DCM three times

Solution yield
82.3%, 74% isolated
assay yield after
silica pad filtration

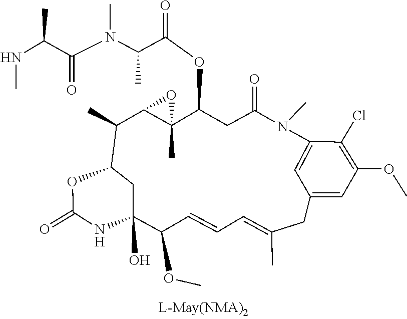

As shown in Table 1, quenching with NH₃/THF proved to be the more desirable method of quench, which affords L-MayNMA in 82% solution assay yield and 74% isolated assay yield after silica pad filtration.

### Example 4. Quench with Sat. NaHCO₃Solution

The reaction conditions for preparing L-MayNMA (Compound Ia) were the same as those described in Example 2. IPC before quench showed that the reaction mixture contained 92.7 area % of L-MayNMA, <5 area % MayOH, and <2 area % of L-May(NMA)₂ and impurities. The reaction mixture was diluted with THF (6 vol w.r.t MayOH) and added sat NaHCO₃. After 3 h, the reaction mixture was sampled and assayed. It was observed that L-May(NMA)₂ increased to 10 area %, L-NCA˜0.4 area %, and MayOH was high at 8 area %. Significant rise in the epimerization to D-MayNMA was also observed. After filtering through celite and concentrating the filtrate to a gummy residue, HPLC assay showed that L-May(NMA)₂ had now dropped to <0.5 area %, and MayOH rose to 18 area %, suggesting that L-May(NMA)₂ had readily hydrolyzed to MayOH, or L-May(NMA)₂ had precipitated out of solution and removed during filtering through celite.

### Example 5. Synthesis of L-MayNMA (Compound Ia) in the Presence of DIPEA Vs Proton Sponge

To a solution of MayOH, DIPEA, and Na₂SO₄ (optional) in DMF under nitrogen was added zinc triflate Zn(OTf)₂ and L-NCA (Compound IIa) at −30° C. and warmed to room temperature. IPC results of the reaction mixture under different conditions were shown in Table 2 below. It was observed that more equivalents of DIPEA caused more epimerization to D-MayNMA.

TABLE 2

| | Reaction | L- | D- | | |
|---|---|---|---|---|---|
| Run | Conditions | MayNMA | MayNMA | Impurities | MayOH |
| 1 | 2 eq L-NCA, 5 | 71% | 9% | 7% | 11% |
| | eq DIPEA, 2 | | | | |
| | eq Zn(OTf)₂, | | | | |
| | No Na₂SO₄, | | | | |
| | 64 h | | | | |
| 2 | 4 eq L-NCA, 12 | 70% | 15% | 4% | 9% |
| | eq DIPEA, 2 | | | | |
| | eq Zn(OTf)₂, | | | | |
| | 6 eq Na₂SO₄, | | | | |
| | 40 h | | | | |
| 3 | 4 eq L-NCA, 12 | 67% | 15% | 2% | 14% |
| | eq DIPEA, 2 | | | | |
| | eq Zn(OTf)₂, | | | | |
| | No Na₂SO₄, | | | | |
| | 40 h | | | | |
| 4 | 2 eq L-NCA, 12 | 33% | 18% | 26% | 16% |
| | eq DIPEA, 4 | | | | |
| | eq Zn(OTf)₂, | | | | |
| | No Na₂SO₄, | | | | |
| | 40 h | | | | |

The reaction procedures for preparing L-MayNMA with proton sponge under different conditions (Table 3) were similar to those described in Example 2. IPC results of the reaction mixture before quench are shown in Table 3.

TABLE 3

| | 1 | 2 | 3 | 4 |
|---|---|---|---|---|
| L-NCA (eq) | 4 | 2 | 2 | 8 |
| H sponge (eq) | 2.75 | 1.5 | 2.75 | 2.75 |
| Zn(OTf)₂ (eq) | 1.5 | 1.5 | 1.5 | 1.5 |
| THF (vol) | 0.6 | 0.4 | 0.6 | 0.6 |
| DMF (vol) | 5.4 | 3.6 | 5.4 | 5.4 |
| L-MayNMA at 24 h IPC | 91.1% | 86.2% | 61.8 | 91.4% |
| L-May(NMA)₂ at 24 h IPC | 0.8% | 0.4% | 0% | 1.9% |
| MayOH at 24 h IPC | 1.6% | 4.4% | 33.0% | 0.7% |

What is claimed is:

1. A method of preparing a compound represented by Formula (I): 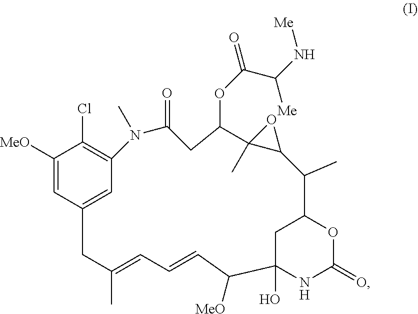 [SMILES: CNC(C)C(=O)OC1CC(=O)N(C)c2cc(cc(OC)c2Cl)C/C(C)=C/C=C/C(OC)C2(O)CC(OC(=O)N2)C(C)C2OC12C]
or a salt thereof, comprising reacting maytansinol with an N-carboxyanhydride in a reaction mixture comprising a base and a Lewis acid to form the compound of Formula (I) or a salt thereof, wherein the N-carboxyanhydride is represented b Formula (II): 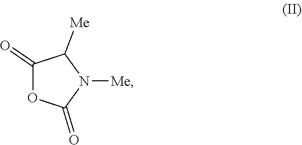
and the base is proton sponge.

2. The method of claim 1, wherein the compound of Formula (I) is represented by Formula (Ia): 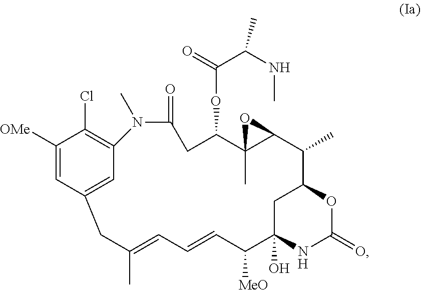 [SMILES: CN[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]
and
the N-carboxyanhydride of Formula (II) is represented by Formula (Ha):

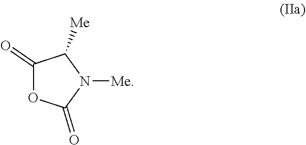

3. The method of claim 1 or 2, wherein the proton sponge is selected from N,N,N′,N′-tetramethyl-1,8-naphthalenediamine, 2,7-dibromo-1,8-bis(dimethylamino)naphthalene (Br₂DMAN), 1,8-bis(hexamethyltriaminophosphazenyl)naphthalene (HMPN), 2,7-di(4-tolylethynyl)-1,8-bis(dimethylamino)naphthalene or its cation, 2,4-bis(trifluoroacetyl)-1,8-bis(dimethylamino)naphthalene, and 2,6-difluoro-1,3,4,5,7,8-hexakis(dimethylamino)naphthalene.

4. The method of claim 3, wherein the proton sponge is N,N,N′N′-tetramethyl-1,8-naphthalenediamine.

5. The method of any one of claims 1-4, wherein the Lewis acid is selected from the group consisting of zinc triflate, zinc chloride, magnesium bromide, magnesium triflate, copper triflate, copper (II) bromide copper (II) chloride, and magnesium chloride.

6. The method of claim 5, wherein the Lewis acid is zinc triflate.

7. The method of any one of claims 1-6, wherein the molar ratio of the Lewis acid to maytansinol is between 0.1:1 to 6:1.

8. The method of claim 7, wherein the molar ratio of the Lewis acid to maytansinol is 1.5:1.

9. The method of any one of claims 1-8, wherein the reaction mixture further comprises a drying agent.

10. The method of claim 9, wherein the drying agent is selected from the group consisting of a molecular sieve, sodium sulfate, calcium sulfate, calcium chloride, and magnesium sulfate.

11. The method of claim 10, wherein the drying agent is sodium sulfate or molecular sieve.

12. The method of claim 11, wherein the drying agent is a molecular sieve.

13. The method of claim 12, wherein the drying agent is in the form of granular beads or powders.

14. The method of any one of claims 1-13, wherein the method further comprises quenching unreacted N-carboxyanhydride by contacting the reaction mixture after the reaction of maytansinol and the N-carboxyanhydride with a solution of ammonia in an organic solvent.

15. The method of claim 14, wherein the reaction mixture is contacted with a solution of ammonia in THF.

16. The method of any one of claims 1-13, wherein the method further comprises reacting unreacted N-carboxyanhydride with a nucleophilic reagent.

17. The method of claim 16, wherein the nucleophilic reagent is water or an alcohol.

18. The method of claim 17, wherein the nucleophilic reagent is water.

19. The method of claim 17, wherein the nucleophilic reagent is an alcohol.

20. The method of claim 19, wherein the alcohol is selected from methanol, ethanol, n-propanol, isopropanol, and tert-butanol.

21. The method of any one of 16-20, wherein the method further comprises contacting the reaction mixture after the reaction of maytansinol and the N-carboxyanhydride with an aqueous solution containing bicarbonate or carbonate or contacting the reaction mixture with a metal scavenger.
